(19) United States
(12) Patent Application Publication
(10) Pub. No.: US 2021/0005312 A1
(43) Pub. Date: Jan. 7, 2021

(54) HEALTH MANAGEMENT SYSTEM WITH MULTIDIMENSIONAL PERFORMANCE REPRESENTATION

(71) Applicant: 3M INNOVATIVE PROPERTIES COMPANY, SAINT PAUL, MN (US)

(72) Inventors: Richard F. Averill, SEYMOUR, CT (US); RICHARD R. FULLER, SCHNECKSVILLE, PA (US); ELIZABETH C. MCCULLOUGH, SILVER SPRING, MD (US); KEITH C. MITCHELL, ROSWELL, GA (US); GARRI L. GARRISON, LEITCHFIELD, KY (US)

(73) Assignee: 3M INNOVATIVE PROPERTIES COMPANY, SAINT PAUL, MN (US)

(21) Appl. No.: 17/025,129

(22) Filed: Sep. 18, 2020

Related U.S. Application Data
(63) Continuation of application No. 16/064,146, filed on Jun. 20, 2018, now abandoned.
(60) Provisional application No. 62/270,735, filed on Dec. 22, 2015.

(51) Int. Cl. G16H 40/20; G06Q 50/22; G06F 17/17; G06Q 10/06

(52) U.S. Cl. CPC G16H 40/20; G06Q 10/0639; G06F 17/175; G06Q 50/22

(57) ABSTRACT

A health management system includes a processor, a searchable multi-dimensional data representation of the performance of an entire health care delivery system accessible by the processor, in which the performance of every healthcare provider, including downstream providers, that are delivering services is distilled down to a clinically credible measure of actual versus expected performance at analytic points across a comprehensive set of quality outcomes and resource utilization measures wherein the performance matrix has multiple dimensions, and a memory device coupled to the processor and having a program stored thereon for execution by the processor to perform operations. The operations include creating the multi-dimensional data representation to obtain performance measures of a selected healthcare provider and accessing the multi-dimensional data representation to obtain performance measures of the selected healthcare provider.

### RELATED APPLICATION

This application claims priority to U.S. Provisional Application Ser. No. 61/271,024 (entitled REAL TIME POPULATION HEALTH MANAGEMENT, filed Dec. 22, 2016) and to U.S. Provisional Application Ser. No. 62/270,735 (entitled HEALTHCARE SYSTEM PERFORMANCE MATRIX AND SEARCH ENGINE, filed Dec. 22, 2016), both of which are incorporated herein by reference.

### BACKGROUND

The implementation of electronic health record systems has increased the volume of data available for healthcare management to the point that it can be overwhelming and often paralyzing. Attempts to find a solution to healthcare management improvement have tended to go in one of two extremes. The first approach is to provide extensive sets of structured comparative reports that the user must search through in order to draw any conclusions and to develop an action plan. The second approach is to use “big data” techniques to search through the vast amounts of data to identify patterns and insights. While the big data approach holds great promise, actual examples of real world operational healthcare problems that have been solved by this approach have been very limited. Furthermore, there is a fundamental difference between identifying a pattern and ultimately finding a solution to the issue identified by the pattern.

### SUMMARY

A health management system includes a processor, a searchable multi-dimensional data representation of the performance of an entire health care delivery system accessible by the processor, in which the performance of every healthcare provider, including downstream providers, that are delivering services is distilled down to a clinically credible measure of actual versus expected performance at analytic points across a comprehensive set of quality outcomes and resource utilization measures wherein the performance matrix has multiple dimensions including individual health care providers, sites of service, quality outcomes and resource use measures, type of patients, time periods covered, geographic location of provider and patient, and the patient's payer, and a memory device coupled to the processor and having a program stored thereon for execution by the processor to perform operations. The operations include creating the multi-dimensional data representation to obtain performance measures of a selected healthcare provider and accessing the multi-dimensional data representation to obtain performance measures of the selected healthcare provider.

A non-transitory machine readable storage device has instructions for execution by a processor of the machine to perform accessing payer data for multiple providers in a health care delivery system, conforming the accessed payer data to a standard format, populating, based on the accessed payer data, a multi-dimensional data representation of the performance of an entire health care delivery system accessible by the processor, in which the performance of every healthcare provider, including downstream providers, that are delivering services is distilled down to a clinically credible measure of actual versus expected performance at analytic points across a comprehensive set of quality outcomes and resource utilization measures wherein the performance matrix has multiple dimensions including individual health care providers, sites of service, quality outcomes and resource use measures, type of patients, time periods covered, geographic location of provider and patient and the patient's payer, creating the multi-dimensional data representation to obtain performance measures of a selected healthcare provider, and accessing the multi-dimensional data representation to obtain performance measures of the selected healthcare provider.

A health management system includes a searchable multi-dimensional data representation of the performance of an entire health care delivery system accessible by one or more processors, in which the performance of every healthcare provider, including downstream providers, that are delivering services, is distilled down to a clinically credible measure of actual versus expected performance at analytic points across a comprehensive set of quality outcomes and resource utilization measures, a memory device coupled to the processor and having a program stored thereon for execution by the one or more processors to perform operations. The operations include creating the multi-dimensional data representation to obtain performance measures of a selected healthcare provider and accessing the multi-dimensional data representation to obtain performance measures of the selected healthcare provider.

### BRIEF DESCRIPTION OF THE DRAWINGS

FIG. 1 is a block diagram representation of a system for integrating information from multiple health care delivery systems to provide a data matrix that is searchable via a search engine according to an example embodiment.

FIG. 2 is a block perspective representation of a three dimensional version of the performance matrix according to an example embodiment.

FIG. 3 is a block schematic flow diagram illustrating population of analytic points in the performance matrix according to an example embodiment.

FIG. 4 is a block diagram of a health management system that includes a real time population health management tool according to an example embodiment.

FIG. 5 is a block diagram of a circuitry adaptable to perform one or more methods and processors with memory according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The rapidly accelerating trend toward provider consolidation and the creation of provider based comprehensive health systems and payment reforms focus on payment bundles such as capitation has created the need for effective population health management. Simultaneously, the implementation of electronic health record systems has increased the volume of data available to the point that it can be overwhelming and often paralyzing. Attempts to find a solution have tended to go in one of two extremes. The first approach is to provide extensive sets of structured comparative reports that the user must search through in order to draw any conclusions and to develop an action plan. The second approach is to use “big data” techniques to search through the vast amounts of data to identify patterns and insights. While the big data approach holds great promise, actual examples of real world operational healthcare problems that have been solved by this approach have been very limited. Furthermore, there is a fundamental difference between identifying a pattern and ultimately finding a solution to the issue identified by the pattern.

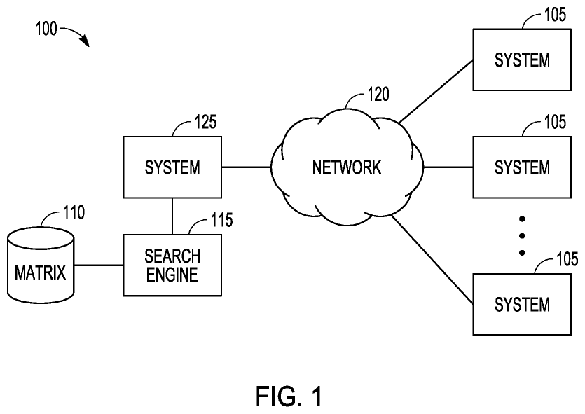

FIG. 1 is a block diagram representation of a system 100 for integrating information from multiple health care delivery systems 105 to provide a data matrix 110 that evaluates performance and is searchable via a search engine 115. The health care delivery systems 105 may be coupled via a network 120 to a system 125 for integration and pre-processing of the data from such health care delivery systems 105 into the matrix 110. System 125 may also be a health care delivery system and include health care data which is also integrated into matrix 110.

In one embodiment, the data matrix is implemented as a performance matrix that is a searchable multi-dimensional data representation of the performance of an entire health care delivery system in which the performance of every healthcare provider who is delivering services is distilled down to a clinically credible measure of actual versus expected performance across a comprehensive set of quality outcomes (readmission rate, complication rate, etc.) and resource use measures (hospital length of stay, pharmaceutical expenditures, etc.). The performance matrix may have multiple dimensions including, but not limited to, individual health care providers, quality outcomes and resource use measures, type of patients, time periods covered, and the patient's payer.

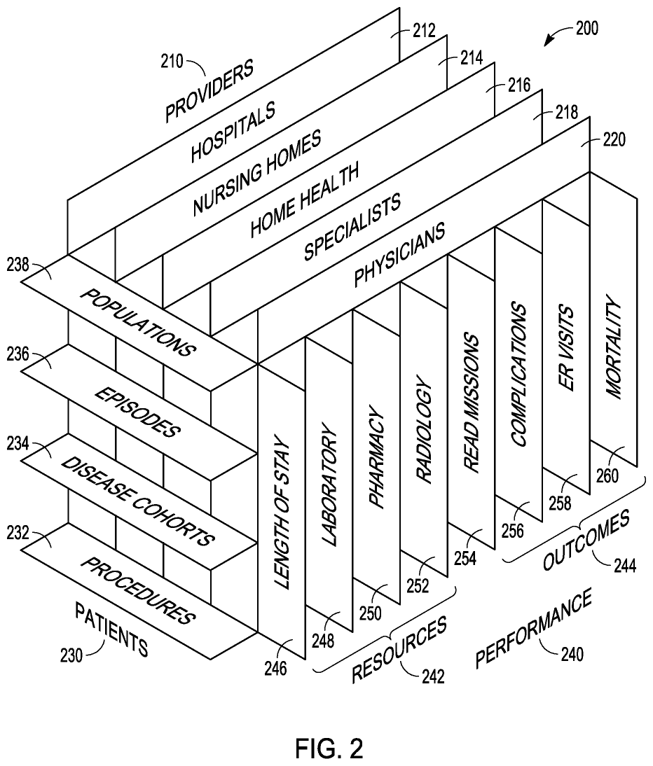

An example representation of a three dimensional version of the performance matrix is shown in a perspective block diagram form in FIG. 2 at 200. The representation may be thought of as a database schema illustrating an overall data base structure comprising multiple analytic points, where each analytic point, also referred to as a cell, incudes actual and expected results of provider performance. In some embodiments, there may be trillions of such analytic points which are in a form that makes it more efficient for a search engine to analyze and derive actual performance results, as well as show areas of performance that are below expected, and why such performance is adversely affected. Such results allow communication of the performance as well as actions that can be taken to improve performance, such as using a different lab for diagnostics, or a different post operation discharge care facility.

The performance matrix 200 represents a new approach that allows the cost and quality performance of an entire health delivery system to be simultaneously evaluated. The performance matrix distills key performance data into an integrated data representation that is searchable allowing the identification of succinct and prioritized information that is clinically credible and at a level of specificity that is actionable and can lead to sustainable behavior changes that lower cost and improve quality.

The performance matrix 200 may be thought of as an integrated data representation that allows the cost and quality performance of an entire health delivery system to be simultaneously evaluated across a multitude of performance measures across all sites of service and providers. The performance matrix distills key performance information into a succinct data representation that is searchable allowing for the identification of information that is at a level of specificity that is actionable and can lead to sustainable behavior changes that lower cost and improve quality.

Matrix 200 includes several dimensions that intersect to form the analytic points. A providers dimension 210 includes hospitals 212, nursing homes 214, home health care 216, specialists 218, and physicians 220. A patients dimension 230 includes procedures 232, disease cohorts 234, episodes 236, and population 238. A performance dimension 240 is broken into a resources portion 242 and outcomes 244. Resources 242 includes length of stay 246, laboratory 248, pharmacy 250, and radiology 252. Outcomes 244 includes readmissions 254, complications 256, emergency room visits 258, and mortality 260.

At its most basic level, excess cost is due to either high unit production cost or an excess volume of services. High or inefficient unit production cost is typically the result of an inability to manage the level of inputs or site of service selection. An excess volume of services is often the result of poor quality since more services will generally be needed to treat the problems caused by the poor quality. To facilitate the development of an action plan to address poor performance, the poor performance needs to be attributed to specific disease categories and specific providers. The performance matrix 200 provides a means of simultaneously evaluating performance across the entire healthcare delivery system. The performance matrix 200, in one embodiment, is a cross tabular representation of the performance of the healthcare delivery system across multiple performance dimensions as previously mentioned, including

Providers or sites of service (hospitals, physicians, specialists, nursing homes, etc.)

Efficiency performance measures (unit expenditures per hospitalization and outpatient visit, per enrollee annual expenditures, expenditures by cost categories such as a laboratory, etc)

Quality performance measures (excess complications, excess readmissions, excess emergency room visits, under-utilization of outpatient mental health services, etc)

Site of service substitution (Over use of skilled nursing facilities versus home health, over utilization of the emergency versus office based primary care, etc)

Expenditure type (total cost of care, individual cost categories such a laboratory, etc.). Expenditure types are only applicable to expenditure performance measures.

Patient Categories (disease cohort such as patients with diabetes, types of encounters such as patients admitted for an appendectomy, etc)

Population segments (total population, disease cohorts, etc.)

Time period (month, year)

Payer (Medicare, Medicaid, commercial insurance company A, insurance company B, etc.)

Geographic location (location of patient, location of site of service, urban/rural, census region, etc)

Individual provider (physician, specialist, hospital, etc)

Thus, the performance matrix has an evaluation of every provider in the healthcare delivery system on every performance measure for every type of expenditure for every population segment for every time period, across a wide range of attributes such as payer and geographic region. For example, the performance matrix includes detailed identification of poor performance such as specifying that the high per patient population expenditures for a primary care physician were due to the high pharmaceutical use by the specialists to whom the primary care physician is referring diabetic patients. Implementations of the performance matrix may be very large, with trillions of analytic points. Each analytic point in the performance matrix contains the following summary performance information that is pre-processed prior to use:

Continuous variables (e.g., expenditures): count, actual average, expected average, test of statistical significance, and binary variables (e.g., readmissions): count, actual rate, expected rate, cost of difference between actual and expected, test of statistical significance.

Thus, each analytic point in the performance matrix contains a pre-processed specific measure of performance expressed as a difference between actual and expected along with the financial impact of the difference. The expected values are risk adjusted to account for differences in case mix. The test of significance provides a determination of whether the observed difference between actual and expect is meaningful (as opposed the result of chance variation). Essentially the Performance Matrix creates a data representation that distills all aspects of delivery system performance down to manageable units of comparison and does every possible drill down providing the basis for identifying the source of performance problems.

Each measure of performance has a pre-computed expected value for every analytic point in the performance matrix. There are many ways to compute an expected value of a performance measure. One of the most common is indirect rate standardization using an exhaustive and mutually exclusive set of risk groups for risk adjustment. Using indirect rate standardization the expected value in the analytic points in the performance matrix is computed based on the following steps:

For each risk group (g) for each performance measure (m), a target value (T(g,m)) is established based on the actual historical average value in a reference database.

For service provider (p) for measure (m). the expected value (E(p,m)) is the sum of overall risk groups of the product of the number of patients/enrollees in each risk group (N(p,m,g) times the corresponding target value (T(g,m) divided by the total number of patients/enrollees:

E(p,m)=sum over g[N(p,m,g)*T(g,m)]/sum over gN(p,m,g)

For service provider (p) for measure (m), the difference between the service provider's actual value and the expected value can be either above expected (negative performance) or below expected (positive performance). Once the Performance Matrix is populated, it is searchable allowing the identification of the sources of poor performance and report the results in a meaningful way that empowers interventions that can lower costs and improve quality.

The performance matrix provides distilled performance down to a financial measure of the difference between actual and expected spending. The financial measures in the performance matrix are essentially a measure of relative internal resource use (production efficiency focusing on volume of services and unit cost). An example of identification of performance differences generated via a search of the performance matrix and presented to the health delivery system is as follows:

In the enrolled population of the health system there are 1,342 patients with CHF (congestive heart failure) who are incurring annual expenditures of $69,752 which is 32 percent higher than would be expected resulting $21.4 million in annual excess expenditures.

80 percent of the excess expenditures are concentrated in high severity CHF patients who have multiple comorbid diseases. The high severity severity CHF patients have a potentially preventable hospital admission rate that is 41 percent higher than expected and a potentially preventable ER visit rate that is 24 percent higher than would be expected.

Although the inpatient hospital expenditures for high severity CHF patients are consistent with expectations the 30 day post-acute care expenditures for these patients are 38 percent higher than would be expected.

52 percent of the excess post-acute care for high severity CHF patients are the result of a potentially preventable readmission rate (that is 62 percent higher than would be expected.

62 percent of the excess post acute care readmission rate is due readmissions from one nursing home (ElderCare) which has a potentially preventable readmission rate that 88 percent higher than would be expected.

78 percent of the patients discharged to this nursing home are for patients discharged by physician James Smith and physician Donald Jones both of whom have a disproportionate number of their high severity CHF patients being discharge to a nursing home.

The overarching objective of the performance matrix is to provide a data model that allows the identification of succinct and prioritized information that is at a level of specificity that is actionable.

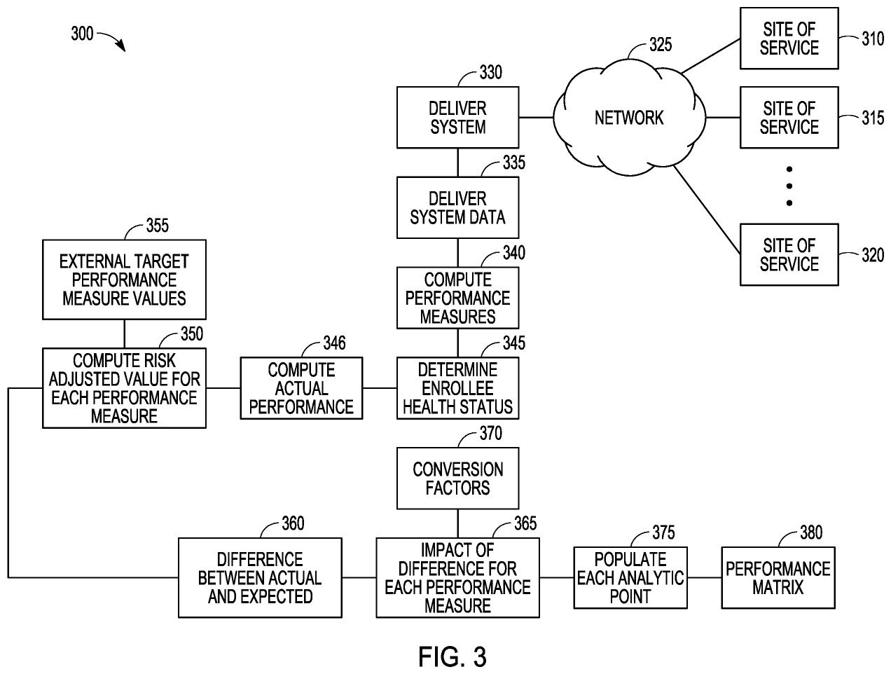

FIG. 3 is a block schematic flow diagram illustrating population of analytic points in the performance matrix generally at 300. Several sites of service are indicated at 310, 315, and 320 coupled by a network 325 to a healthcare delivery system 330. Sites of service 310, 315, and 320 may be downstream providers which each have their own health care databases with information regarding patients and services provided, as well as performance data, medical records, and other information. System 330 has longitudinally integrated delivery system data 335 that represents all information regarding healthcare services provided by healthcare providers covered by system 330. The data 335 may be gathered from multiple different databases for the delivery system, but provides a consistent interface to that data.

At 340, processing is performed on the data to computer performance measures. Enrollee health status is determined at 345. In one embodiment, the enrollee corresponds to a patient receiving services at delivery system 330 and the various network coupled sites of service. At 350, a risk adjusted expected value for each performance measure is computed. The risk adjusted expected value may include external target performance measure values 355, corresponding to the networked connected sites of service 310, 315, and 320.

A difference between actual and expected value for each performance measure is calculated at 365 and may include conversion factors 370 to convert data from the connected sites of service 310, 315, and 320 that may not be stored using the same schema as data 335, which may be a canonical form of data. In some embodiments, both data 335 and data from the connected sites of service may be converted to a canonical form.

In one embodiment, the difference between actual and expected value for each performance measure is a representation of the impact, such as a financial impact for each performance measure. At 375, the impact from 365 is used to populate each analytic point or cell in the performance matrix 200, resulting in a completed performance matrix 380 ready for use.

In one embodiment, longitudinal historical claims data, such as data from one or more insurance companies (payer) for multiple patients and multiple providers is obtained at 335 from one or more systems. The data obtained may be run through a classification system to obtain a consistent representation of the data at 340, 345 and define what each service corresponding to the claims was. One example classification system includes a 3M Patient Classification System. The data may be used to determine the actual performance at 346. The classification data from classification 340, 345 is also used to generate performance norms for quality outcomes and resource use at 355. At 360, the actual and expected performance is compared to generate performance differences by subtracting the actual performance measure from the expected performance. The result is used to determine the financial impact of negative quality outcomes at 365, which may involve aggregating data from multiple patients over multiple providers and other dimensions. This information is then used to populate the performance matrix at 380.

In various embodiments, the use of the performance matrix may provide for real time population health care management. As the healthcare industry moves towards increasing use of Accountable Care Organizations (ACOS) and the shift to bundled payment (meaning a single payment to cover all aspects of care for a given condition), there is an increased need for tools to actively manage the healthcare of populations of patients across a wider range of settings and contexts. This management extends beyond those times where the patient is an admitted patient or in the provider's office for a visit to include factors such as but not limited to prescription compliance, preventative checkups, preventative vaccinations, healthy living activities, and living arrangements such as assisted living centers, etc. Both private and public healthcare payers increasingly mandate sets of care guidelines and criteria that need to be followed by providers. If they are not followed, providers may not be fully reimbursed for services provided, patient care may be adversely affected, and the overall health of the patient population may be less than optimal.

In many cases, healthcare provider organizations are required to not only manage adherence to such care guidelines on a per patient level, but also to report their compliance at a population level to various payers and government health agencies. Typically, in the industry today this is a time consuming process that requires a significant amount of manual effort to complete. Determining whether or not provided care is within appropriate guidelines requires the review of a wide range of data sources including but not limited to the Electronic Health Records, Visit Scheduling information, Lab and Diagnostic reports, Pharmacy data, and even a patient's own health tracking data. The process of bringing such data sets together for complete review is usually a cumbersome one. Timing of access to data sets, for one thing, can be an issue: not all cases are usually able to be reviewed in time for interventions to correct cases where proper guidelines are not followed as the reviews are often retrospective to the patient having left the hospital or provider. For the provider organization this can result in costly claims denials or loss of reimbursement, and for the patient it can result in sub-optimal health treatments when, for example, an incorrect site of service is selected, necessary diagnostics are not performed, diagnostics are performed unnecessarily, medications are not filled and used by the patient, and so on.

Many of the challenges associated with beginning to manage care in this new way come from data being housed in multiple systems that are not integrated and which span organizational boundaries. A full review of patient care from all settings requires knowledge of multiple systems, review of paper documentation, review of visit schedules, development of a longitudinal view of a patient and their associated health issues, and then tracking and coordinating that patient's care in accordance with the necessary guidelines across this myriad of systems.

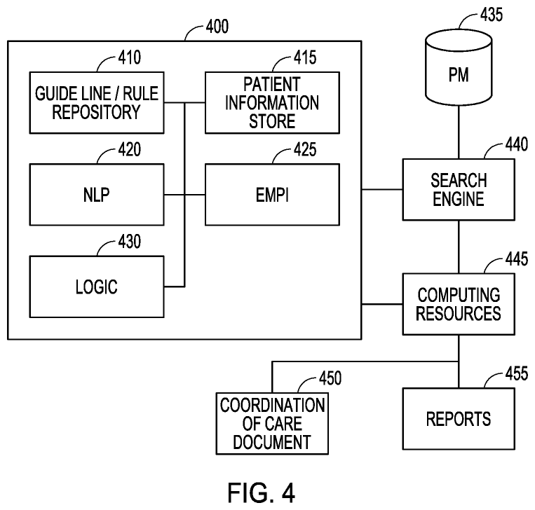

FIG. 4 is a block diagram of a health management system 400 that includes a real time population health management tool 405 to improve an organization's ability to care for its population of patients while simultaneously reducing the manual efforts required to do so and enabling better use of the organization's resources to focus on the delivery of proper care. The tool in one embodiment is implemented in software for execution on a processor in a local or cloud computing environment.

Tool 405 includes several components, including but not limited to a guideline/rule repository 410, a patient information store 415, natural language processing (NLP) 420, enterprise master person index (EMPI) 425, and criteria evaluation logic 430. The tool 405 also has access to a performance matrix 435 and performance matrix search engine 440. The components may execute on the search engine 440, or other local or remote processing resources 445, or a combination thereof.

Guideline/rule repository 410 contains rule sets needed to satisfy a given care protocol, reporting guideline, or compliance standard. These may apply at a particular patient or population level. Examples of these include Core Measures, Patient Safety Incidents, Hospital Acquired Conditions/Infections, Preventable Complication or Readmission Requirements, Site of Service assignment criteria, criteria in determining patient transportation, patient placement, and care criteria for specific disease, condition, or risk cohorts.

Patient Information Store 415 is a repository that contains the universe of data known about a specific patient. It extends beyond just data that is available in the Electronic Health Record to include information such as scheduled care follow ups, prescription refill information, diagnostics ordered, and patient captured data such as glucose monitoring information. The term “Patient Information Store” is a generic term for this collection of data as in reality the store may actually be comprised of multiple repositories able to be accessed collectively, to assemble the total longitudinal picture of a patient's health care information. Data elements may be populated via direct interface with structured data from other systems and may be represented in a variety of formats or code sets such as ICD9, ICD10, SNOMED-CT, LOINC, etc. Unstructured data in the Patient Information Store may be processed using Natural Language Processing (NLP) to extract clinical facts from text narrative and other unstructured data sources. In one embodiment, the data is aggregated from a variety of care settings, and includes financial data, patient tracked data, and disease specific items. All data elements are represented with unique concept identifiers that are in turn mapped to care guidelines and rules that makes use of particular types of data. The concept identifiers may be combined to construct a longitudinal patient problem list and care history, which may be compared to relevant care guidelines for patients based on plan membership, quality reporting guidelines, and other factors.

Natural language processing (NLP) 420 component is used to extract data, including clinical facts, from semi-structured and unstructured data sources. The NLP also maps the clinical facts found in those sources to discrete elements of the data sources needed to evaluate against rules. Also used to facilitate the question/answer process needed to query the longitudinal patient record as updates are made which affect the Coordination of Care document.

Enterprise Master Person Index (EMPI) 425 is used to consolidate data from various systems and sources around a single patient record. Includes ability to match patient data from systems using identifiers from systems and other identifying information such as Date of Birth, Government ID numbers, Insurance Identifiers, etc. Several vendors provide the ability to match patient data based on multiple, such as 12 or more such pieces of information to provide an assurance that patients are correctly identified and their corresponding data is accurate.

Criteria evaluation logic 430 is used to apply sets of care guidelines and criteria to the data for a particular patient to determine which have been satisfied and which are deficient. Operationalizes the Guideline/Rule Repository and the Patient Information Store together to produce data for the system outputs. Compares data for patient being evaluated against outcomes for similar patients (based on available data elements) to offer insights into likely successful care steps. Considers output of tools such as the performance matrix which will inform the evaluation of next care steps for the patient against the current state of the health system's ability to successfully deliver those steps. Care deficiencies and needed care may be identified and prioritized.

The tool 405 may take a variety of different types of patient health data as input. While the more available data, the more complete the tool's review and recommendations will be, not all data sources are required for the Tool to provide valuable feedback. Types of data that the Tool may make use of include but are not limited to: patient claims data, pharmacy/medication refill data, pre/post hospital care setting data, clinical documents, visit scheduling information, and personal health information tracked by the patient (e.g. weights, blood pressure, glucose information, exercise data).

The tool 405 will initially enable two primary outputs. One output is a Coordination of Care Document 450. As new clinical documents and diagnostic information about a patient becomes available to the tool, the system evaluates the new data against any known care guidelines that apply to the patient based on the patient's existing health conditions. The tool updates any criterion met by the new data and identifies any new deficiencies that may be introduced by the new data. For example, a particular result on one diagnostic test may warrant a next test be conducted; or the completion of one type of follow up or preventative visit will then trigger the next required visit to be determined.

The new data will also be evaluated to determine if it warrants adding the patient to new care guideline groups. Adding the patient to care guideline groups may be done automatically by the Tool, either by the Tool itself or by the Tool calling a sub-process in another system; or the Tool may flag the record for evaluation by a human reviewer who may add the patient to the new group. This may occur for example if the new incoming data suggests or definitively diagnoses a new disease such as diabetes. The system will evaluate the known data about the patient against the new care guideline membership as a diabetic, indicate the initial care steps that need to be applied to the patient, and also flag the patient for inclusion in any reporting on the population of diabetic patients.

The Coordination of Care Document 450 is accessible by users of the system such as providers and Care Managers as needed through a user interface as is commercially available, such as the 360 Encompass MD user interface provided by 3M Health Information Systems.

Users will also have the ability to request that the system update the record in “real-time” if needed to incorporate newly added data elements and receive immediate feedback on additional care suggestions or necessary steps to take with the patient. This might also occur for example when a patient currently being seen in the Emergency Department needs to be evaluated against criteria for assignment to a particular site of service or against inpatient admission criteria.

The Coordination of Care Document 450 will offer prioritized guidance for necessary care that is informed by analyzing outcomes of care for patients deemed to be similar to a particular patient based on available data elements within the population. Prioritization will also incorporate feedback from tools such as the 3M Health System Performance Matrix, which can assist in prioritizing care options based on current performance of the healthcare delivery system itself. This guidance may also include querying the clinical records of the population using NLP in addition to structured/coded data—e.g. to generate ad-hoc population information relevant to the current patient based on patient specific characteristics.

In one embodiment, prioritized worklists may be presented for individual patients. Prioritization may be informed by outcome data from a population of like patients within populations.

Reports 455 on Extracted Data may also be provided as an output. The system may generate reports on a scheduled basis for measures identified by different care guideline groups. Examples of this would include reporting to national or state quality agencies, compliance with care protocols for particular diseases, effectiveness of preventative care measures, rates of compliance with prescription medication refills, etc. Automated reporting of population care delivered versus care guidelines may be generated.

Care Managers may also see a prioritized list of patients within their population in varying states of care that need attention to stay within care guidelines. Examples of this would be: all patients currently admitted within the healthcare system, all patients due for a particular type of follow up visit, call or diagnostic, or patients needing follow up on medication refills. A prioritized worklist may also be generated for an overall population.

Anticipated benefits to users of the system, depending on implementation, may include a reduction in manual effort required to do mandated reporting, which would in turn enable cost savings or redeployment of resources to more directly affect patientcare. A further benefit may include an increase of case review for compliance with varying care guidelines from current percentage to 100%. A reduction in denials, reduction in Recovery Audit Contractor (RAC) audits, reduction in payment penalties related to: readmissions, hospital acquired conditions, patient safety indicators, and lost reimbursement due to issues such as incorrect site of service assignment, patients not meeting admission criteria. An Improved ability may be provided to produce prioritized lists of patients at risk for not meeting care guidelines based on specific disease conditions (e.g. diabetes, heart disease) or other criteria. Yet a further benefit may include an improved ability to predict future care needs of population based on a more comprehensive review of population status. The tool may further provide for integration of population management into a single workflow within a single system rather than many disparate systems. Overall, a reduction in complexity of care management process may also be provided.

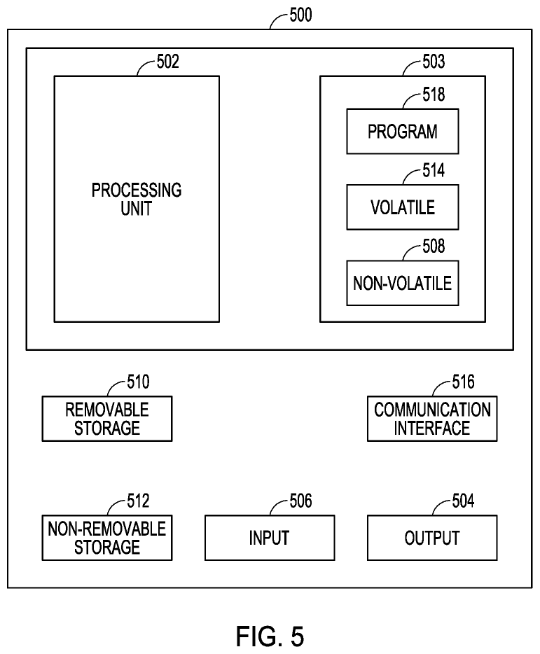

FIG. 5 is a block schematic diagram of a computer system 500 to implement methods according to example embodiments. All components need not be used in various embodiments. One example computing device in the form of a computer 500, may include a processing unit 502, memory 503, removable storage 510, and non-removable storage 512. Although the example computing device is illustrated and described as computer 500, the computing device may be in different forms in different embodiments. For example, the computing device may instead be a smartphone, a tablet, smartwatch, or other computing device including the same or similar elements as illustrated and described with regard to FIG. 5. Devices such as smartphones, tablets, and smartwatches are generally collectively referred to as mobile devices. Further, although the various data storage elements are illustrated as part of the computer 500, the storage may also or alternatively include cloud-based storage accessible via a network, such as the Internet.

Memory 503 may include volatile memory 514 and non-volatile memory 508. Computer 500 may include—or have access to a computing environment that includes—a variety of computer-readable media, such as volatile memory 514 and non-volatile memory 508, removable storage 510 and non-removable storage 512. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices capable of storing computer-readable instructions for execution to perform functions described herein.

Computer 500 may include or have access to a computing environment that includes input 506, output 504, and a communication connection 516. Output 504 may include a display device, such as a touchscreen, that also may serve as an input device. The input 506 may include one or more of a touchscreen, touchpad, mouse, keyboard, camera, one or more device-specific buttons, one or more sensors integrated within or coupled via wired or wireless data connections to the computer 500, and other input devices. The computer may operate in a networked environment using a communication connection to connect to one or more remote computers, such as database servers, including cloud based servers and storage. The remote computer may include a personal computer (PC), server, router, network PC, a peer device or other common network node, or the like. The communication connection may include a Local Area Network (LAN), a Wide Area Network (WAN), cellular, WiFi, Bluetooth, or other networks.

Computer-readable instructions stored on a computer-readable storage device are executable by the processing unit 502 of the computer 500. A hard drive, CD-ROM, and RAM are some examples of articles including a non-transitory computer-readable medium such as a storage device. The terms computer-readable medium and storage device do not include carrier waves. For example, a computer program 518 may be used to cause processing unit 502 to perform one or more methods or algorithms described herein.

### EXAMPLES

In example 1, a health management system includes a processor, a searchable multi-dimensional data representation of the performance of an entire health care delivery system accessible by the processor, in which the performance of every healthcare provider, including downstream providers, that are delivering services is distilled down to a clinically credible measure of actual versus expected performance at analytic points across a comprehensive set of quality outcomes and resource utilization measures wherein the performance matrix has multiple dimensions including individual health care providers, sites of service, quality outcomes and resource use measures, type of patients, time periods covered, geographic location of provider and patient, and the patient's payer, and a memory device coupled to the processor and having a program stored thereon for execution by the processor to perform operations. The operations include creating the multi-dimensional data representation to obtain performance measures of a selected healthcare provider and accessing the multi-dimensional data representation to obtain performance measures of the selected healthcare provider.

Example 2 includes the health management system of example 1 wherein the clinically credible measure comprises at least one of readmission rate and complication rate.

Example 3 includes the health management system of any of examples 1-2 wherein the healthcare providers include at least multiple of hospitals, nursing homes, home health care agencies, specialists, and physicians.

Example 4 includes the health management system of any of examples 1-3 wherein the types of patients include at least one of encounters for a procedure, encounters for chronic or acute disease management, disease cohorts of patients, episodes of care, and population management.

Example 5 includes the health management system of any of examples 1-4 wherein a performance dimension of the performance matrix is broken into a resources portion and a quality outcomes portion.

Example 6 includes the health management system of example 5 wherein the resource portions includes at least one of length of stay, laboratory, pharmacy, and radiology.

Example 7 includes the health management system of any of examples 5-6 wherein the outcomes portion includes at least one of readmissions, complications, emergency room visits, and mortality.

Example 8 includes the health management system of any of examples 1-7 wherein each analytic point in the performance matrix contains a pre-processed specific measure of performance expressed as a difference between actual and expected along with the financial impact of the difference.

Example 9 includes the health management system of example 8 wherein expected values are risk adjusted to account for differences in case mix.

Example 10 includes the health management system of any of examples 8-9 wherein the pre-processed specific measure of performance of each analytic point is pre-calculated using indirect rate standardization based on an exhaustive and mutually exclusive set of risk groups for risk adjustment.

Example 11 includes the health management system of example 10 wherein for each risk group (g) for each performance measure (m), a target value (T(g,m)) is established based on an actual historical average value in a reference database.

Example 12 includes the health management system of example 11 wherein for service provider (p) for measure (m), an expected value (E(p,m)) is the sum of overall risk groups of the product of the number of patients/enrollees in each risk group (N(p,m,g) times the corresponding target value (T(g,m) divided by the total number of patients/enrollees expressed as: E(p,m)=sum over g[N(p,m,g)*T(g,m)]/sum over gN(p,m,g), and wherein the difference between the service provider's actual value and the expected value is expressed as above expected (negative performance) or below expected (positive performance).

In example 13, a non-transitory machine readable storage device has instructions for execution by a processor of the machine to perform accessing payer data for multiple providers in a health care delivery system, conforming the accessed payer data to a standard format, populating, based on the accessed payer data, a multi-dimensional data representation of the performance of an entire health care delivery system accessible by the processor, in which the performance of every healthcare provider, including downstream providers, that are delivering services is distilled down to a clinically credible measure of actual versus expected performance at analytic points across a comprehensive set of quality outcomes and resource utilization measures wherein the performance matrix has multiple dimensions including individual health care providers, sites of service, quality outcomes and resource use measures, type of patients, time periods covered, geographic location of provider and patient and the patient's payer, creating the multi-dimensional data representation to obtain performance measures of a selected healthcare provider, and accessing the multi-dimensional data representation to obtain performance measures of the selected healthcare provider.

Example 14 includes the non-transitory machine readable storage device of example 13 wherein the clinically credible measure comprises at least one of readmission rate and complication rate.

Example 15 includes the non-transitory machine readable storage device of any of examples 13-14 wherein the healthcare providers include at least multiple of hospitals, nursing homes, home health care agencies, specialists, and physicians.

Example 16 includes the non-transitory machine readable storage device of any of examples 13-15 wherein the types of patients include at least one of encounters for a procedure, encounters for chronic or acute disease management, disease cohorts of patients, episodes of care, and population management.

Example 17 includes the non-transitory machine readable storage device of any of examples 13-16 wherein a performance dimension of the performance matrix is broken into a resources portion and an quality outcomes portion.

Example 18 includes the non-transitory machine readable storage device of example 17 wherein the resource portions include at least one of length of stay, laboratory, pharmacy, and radiology.

Example 19 includes the non-transitory machine readable storage device of any of examples 17-18 wherein the outcomes portion includes at least one of readmissions, complications, emergency room visits, and mortality.

Example 20 includes the non-transitory machine readable storage device of example 13 wherein each analytic point in the performance matrix contains a pre-processed specific measure of performance expressed as a difference between actual and expected along with the financial impact of the difference.

Example 21 includes the non-transitory machine readable storage device of example 20 wherein expected values are risk adjusted to account for differences in case mix.

Example 22 includes the non-transitory machine readable storage device of any of examples 20-21 wherein the pre-processed specific measure of performance of each analytic point is pre-calculated using indirect rate standardization based on an exhaustive and mutually exclusive set of risk groups for risk adjustment.

Example 23 includes the non-transitory machine readable storage device of example 22 wherein for each risk group (g) for each performance measure (m), a target value (T(g,m)) is established based on an actual historical average value in a reference database.

Example 24 includes the non-transitory machine readable storage device of example 23 wherein for service provider (p) for measure (m), an expected value (E(p,m)) is the sum of overall risk groups of the product of the number of patients/enrollees in each risk group (N(p,m,g) times the corresponding target value (T(g,m) divided by the total number of patients/enrollees expressed as:

E(p,m)=sum over g[N(p,m,g)*T(g,m)]/sum over gN(p,m,g), and wherein the difference between the service provider's actual value and the expected value is expressed as above expected (negative performance) or below expected (positive performance).

In example 25, a health management system includes a searchable multi-dimensional data representation of the performance of an entire health care delivery system accessible by one or more processors, in which the performance of every healthcare provider, including downstream providers, that are delivering services, is distilled down to a clinically credible measure of actual versus expected performance at analytic points across a comprehensive set of quality outcomes and resource utilization measures, a memory device coupled to the processor and having a program stored thereon for execution by the one or more processors to perform operations. The operations include creating the multi-dimensional data representation to obtain performance measures of a selected healthcare provider and accessing the multi-dimensional data representation to obtain performance measures of the selected healthcare provider.

Example 26 includes the health management system of example 25 wherein the clinically credible measure comprises at least one of readmission rate and complication rate.

Example 27 includes the health management system of any of examples 25-26 wherein the performance matrix has multiple dimensions including individual health care providers, sites of service, quality outcomes and resource use measures, type of patients, time periods covered, geographic location of provider and patient and the patient's payer, wherein the healthcare providers include at least multiple of hospitals, nursing homes, home health care agencies, specialists, and physicians.

Example 28 includes the health management system of example 27 wherein the types of patients include at least one of encounters for a procedure, encounters for chronic or acute disease management, disease cohorts of patients, episodes of care, and population management.

Example 29 includes the health management system of any of examples 27-28 wherein a performance dimension of the performance matrix is broken into a resources portion and an outcomes portion.

Example 30 includes the health management system of example 29 wherein the resource portions include at least one of length of stay, laboratory, pharmacy, and radiology.

Example 31 includes the health management system of any of examples 29-30 wherein the outcomes portion includes at least one of readmissions, complications, emergency room visits, and mortality.

Example 32 includes the health management system of any of examples 25-31 wherein each analytic point in the performance matrix contains a pre-processed specific measure of performance expressed as a difference between actual and expected along with the financial impact of the difference.

Example 33 includes the health management system of example 32 wherein expected values are risk adjusted to account for differences in case mix.

Example 34 includes the health management system of any of examples 32-33 wherein the pre-processed specific measure of performance of each analytic point is pre-calculated using indirect rate standardization based on an exhaustive and mutually exclusive set of risk groups for risk adjustment.

Example 35 includes the health management system of example 34 wherein for each risk group (g) for each performance measure (m), a target value (T(g,m)) is established based on an actual historical average value in a reference database.

Example 36 includes the health management system of example 35 wherein for service provider (p) for measure (m), an expected value (E(p,m)) is the sum of overall risk groups of the product of the number of patients/enrollees in each risk group (N(p,m,g) times the corresponding target value (T(g,m) divided by the total number of patients/enrollees expressed as: E(p,m)=sum over g[N(p,m,g)*T(g,m)]/sum over gN(p,m,g), and wherein the difference between the service provider's actual value and the expected value is expressed as above expected (negative performance) or below expected (positive performance).

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1-15. (canceled)

16. A health care management system comprising:
a processor communicatively coupled to a computing network;
a memory device, coupled to the processor, storing a searchable multi-dimensional data representation of the performance of an entire health care delivery system accessible by the processor, in which the performance of each healthcare provider represented by the multi-dimensional data representation, including downstream providers, that are delivering services is distilled down to a clinically credible measure of actual versus expected performance at analytic points across one or more quality outcomes and one or more resource utilization measures, wherein the multi-dimensional data representation is searchable to obtain performance measures of a selected healthcare provider via a query identifying the selected healthcare provider, wherein the memory device has a program stored thereon for execution by the processor to perform operations comprising:
generating the multi-dimensional data representation by receiving information from multiple health care providers via the computing network, preprocessing the information, and storing the preprocessed information into the multi-dimensional data representation;
receiving input requesting one or more suggestions for the provision of healthcare services to provide to a first patient;
accessing the multi-dimensional data representation and comparing the first patient to one or more other patients represented by the multi-dimensional data representation to determine which one or more second patients are similar to the first patient, wherein similarity is determined automatically based on a comparison of one or more characteristics defined by one or more data elements in the multi-dimensional data representation that are shared by both the first patient and the one or more second patients;
analyzing outcomes of care pertaining to potential healthcare services for the first patient with outcomes of care pertaining to healthcare services previously administered to the one or more second patients;
determining a prioritized guidance for the provision of additional healthcare services based on the analysis; and
generating a report that includes the prioritized guidance for the provision of additional healthcare services for the first patient.

17. The health management system of claim 16, wherein a performance dimension of the multi-dimensional data representation is sub-divided into a resources dimension and a quality outcomes dimension.

18. The health management system of claim 17, wherein the resources dimension includes at least one of length of stay, laboratory utilization, pharmacy utilization, and radiology utilization, and wherein the outcomes dimension includes at least one of readmissions, complications, emergency room visits, and mortality.

19. The health management system of claim 16, wherein each analytic point in the multi-dimensional data representation contains a pre-computed specific measure expressed as a difference between actual and expected performance, wherein the expected values are risk adjusted to account for differences in case mix, and wherein the pre-processed specific measure of performance of each analytic point is pre-calculated using indirect rate standardization based on an exhaustive and mutually exclusive set of risk groups for risk adjustment.

20. The health care management system of claim 19, wherein the pre-computed specific measure further comprises financial impact of the difference between the actual and expected performance.

21. The health management system of claim 19, further comprising a reference database stored on the memory device, wherein for each risk group (g) for each clinically credible performance measure (m), a target value (T(g,m)) is established based on an actual historical average value in the reference database of patients and enrollees of a service provider (p) for whom behavior change by the service provider (p) is possible, and wherein for service provider (p) for measure (m), an expected value (E(p,m)) is the sum of overall risk groups of the product of the number of patients and enrollees patients and enrollees of the service provider (p) for whom behavior change by the service provider (p) is possible in each risk group (N(p,m,g) times the corresponding target value (T(g,m) divided by the total number of patients and enrollees patients and enrollees of a service provider (p) for whom behavior change by the service provider (p) is possible expressed as:
E(p,m)=sum over g[N(p,m,g)*T(g,m)]/sum over gN(p,m,g) and wherein the difference between the service provider's actual value and the expected value is expressed as above expected (negative performance) or below expected (positive performance).

22. The health management system of claim 16, further comprising:
a rule repository stored on the memory device, wherein the operations further comprise:
accessing one or more rules stored in the rule repository;
evaluating, using the one more rules, a collection of care guidelines and criteria; and
determining, based upon the evaluating, which care guidelines are satisfied and which care guidelines are deficient.

23. The health management system of claim 22, wherein determining a prioritized guidance for the provision of additional healthcare services is based on determining which care guidelines are deficient.

24. The health management system of claim 22, wherein the operations further comprise:
storing the prioritized guidance for the provision of additional healthcare services on the memory device;
receiving additional information concerning the provision of care for the first patient;
accessing the stored prioritized guidance;
analyzing outcomes of care pertaining to potential healthcare services for the first patient with outcomes of care pertaining to healthcare services previously administered to the one or more second patients;
determining a second prioritized guidance for the provision of additional healthcare services based on the analysis; and
generating a second report that includes the prioritized guidance for the provision of additional healthcare services for the first patient.

25. The health management system of claim 24, wherein the additional information comprises new clinical documents and diagnostic information concerning the first patient.

26. A computer-implemented method for generating prioritized guidance for the provision of additional healthcare services, comprising:
accessing payer data for multiple providers in a health care delivery system via a network;
conforming the accessed payer data to a standard format;
generating, based on the accessed payer data, a searchable multi-dimensional data representation of the performance of an entire health care delivery system accessible by the processor, in which the performance of each healthcare provider represented by the multi-dimensional data representation, including downstream providers, that are delivering services is distilled down to a clinically credible measure of actual versus expected performance at analytic points across one or more quality outcomes and one or more resource utilization measures, wherein the multi-dimensional data representation is searchable to obtain performance measures of a selected healthcare provider via a query identifying the selected healthcare provider;
storing the searchable multi-dimensional data representation;
receiving input requesting one or more suggestions for the provision of healthcare services to provide to a first patient;
accessing the stored multi-dimensional data representation and comparing the first patient to one or more other patients represented by the multi-dimensional data representation to determine which one or more second patients are similar to the first patient, wherein similarity is determined automatically based on a comparison of one or more characteristics defined by one or more data elements in the multi-dimensional data representation that are shared by both the first patient and the one or more second patients;
analyzing outcomes of care pertaining to potential healthcare services for the first patient with outcomes of care pertaining to healthcare services previously administered to the one or more second patients;
determining a prioritized guidance for the provision of additional healthcare services based on the analysis; and
generating a report that includes the prioritized guidance for the provision of additional healthcare services for the first patient.

27. The computer-implemented method of claim 26, wherein a performance dimension of the multi-dimensional data representation is sub-divided into a resources dimension and a quality outcomes dimension.

28. The computer-implemented method of claim 27, wherein the resources dimension includes at least one of length of stay, laboratory utilization, pharmacy utilization, and radiology utilization, and wherein the outcomes dimension includes at least one of readmissions, complications, emergency room visits, and mortality.

29. The computer-implemented method of claim 26, wherein each analytic point in the multi-dimensional data representation contains a pre-computed specific measure expressed as a difference between actual and expected performance, wherein the expected values are risk adjusted to account for differences in case mix, and wherein the pre-processed specific measure of performance of each analytic point is pre-calculated using indirect rate standardization based on an exhaustive and mutually exclusive set of risk groups for risk adjustment.

30. The computer-implemented method of claim 29, wherein the pre-computed specific measure further comprises financial impact of the difference between the actual and expected performance.

31. The computer-implemented method of claim 29, further comprising:
accessing a reference database that contains one or more historical averages of one or more clinically credible performance measures; and
determining one or more expected values for one or more clinically credible performance measures, wherein for each risk group (g) for each clinically credible performance measure (m), a target value (T(g,m)) is established based on an actual historical average value in the reference database of patients and enrollees of a service provider (p) for whom behavior change by the service provider (p) is possible, and wherein for service provider (p) for measure (m), an expected value (E(p,m)) is the sum of overall risk groups of the product of the number of patients and enrollees patients and enrollees of the service provider (p) for whom behavior change by the service provider (p) is possible in each risk group (N(p,m,g) times the corresponding target value (T(g,m) divided by the total number of patients and enrollees patients and enrollees of a service provider (p) for whom behavior change by the service provider (p) is possible expressed as:
E(p,m)=sum over g[N(p,m,g)*T(g,m)]/sum over gN(p,m,g) and wherein the difference between the service provider's actual value and the expected value is expressed as above expected (negative performance) or below expected (positive performance).

32. The computer-implemented method of claim 26, further comprising:
accessing one or more rules stored in the rule repository;
evaluating, using the one more rules, a collection of care guidelines and criteria; and
determining, based upon the evaluating, which care guidelines are satisfied and which care guidelines are deficient.

33. The computer-implemented method of claim 32, wherein determining a prioritized guidance for the provision of additional healthcare services is based on determining which care guidelines are deficient.

34. The computer-implemented method of claim 32, further comprising:
storing the prioritized guidance for the provision of additional healthcare;
receiving additional information concerning the provision of care for the first patient;
accessing the stored prioritized guidance;
analyzing outcomes of care pertaining to potential healthcare services for the first patient with outcomes of care pertaining to healthcare services previously administered to the one or more second patients;
determining a second prioritized guidance for the provision of additional healthcare services based on the analysis; and
generating a second report that includes the prioritized guidance for the provision of additional healthcare services for the first patient.

35. The computer-implemented method of claim 34, wherein the additional information comprises new clinical documents and diagnostic information concerning the first patient.
